(19) United States
(12) Patent Application Publication
(10) Pub. No.: US 2010/0196497 A1
(43) Pub. Date: Aug. 5, 2010

(54) Method of Treating Tissue Using Platelet-Rich Plasma in Combination with Low-Level Laser Therapy

(75) Inventors: Susan M.L. LIM, Singapore (SG); Steven C. SHANKS, McKinney, TX (US); Ryan MALONEY, Phoenix, AZ (US)

Correspondence Address: ETHERTON LAW GROUP, LLC, 2010 E. University Drive, Suite 25, Tempe, AZ, 85281, US

(73) Assignee: THERAPY PRODUCTS, INC., McKinney, TX (US)

(21) Appl. No.: 12/698,706

(22) Filed: Feb. 2, 2010

Related U.S. Application Data
(60) Provisional application No. 61/149,055, filed on Feb. 2, 2009.

(51) Int. Cl. A61K 35/16; A61P 19/04; A61P 3/10

(52) U.S. Cl. 424/530

(57) ABSTRACT

This invention is a method of treating a patient's injured tissue by applying platelet-rich plasma and laser energy to the injured tissue. The applied energy is low-level, and the patient feels no sensation of the low-level laser energy being applied. The laser energy can be applied before, after, or during the platelet-rich plasma application, or any combination thereof. Additional laser therapy may be applied over the entire extremity containing the injury, any non-injured adjacent tissue, as well as to the patient's entire body for stimulation of other body systems. Additionally, laser energy can be applied to the platelet-rich plasma before the platelet-rich plasma is applied to the patient.

### CROSS-REFERENCE TO RELATED APPLICATIONS

This application claims the benefit of co-pending provisional application No. 61/149,055 filed Feb. 2, 2009.

### FIELD OF INVENTION

This invention relates generally to tissue repair. This invention relates particularly to the application of platelet-rich plasma and low-level laser energy for treating dermal and musculoskeletal tissue injuries.

### BACKGROUND

Platelets serve a diverse role in the human body with their most important function facilitating wound healing and hemostasis. The repair response of tissue, whether dermal or musculoskeletal, generally starts with the formation of a blood clot and degranulation of platelets, which releases growth factors and cytokines at the site of injury. The wound bed passively absorbs the growth factors which then stimulate the fibroblasts within the wound bed to proliferate and themselves secrete further cytokines as well as produce collagen.

The release of cytokines via platelets increases integrins on fibroblasts and epithelial cells, promoting their activation and proliferation. Platelets adhere to exposed matrix via integrins that bind to collagen and laminin. Kertainocytes migrate into the area and begin the restoration process of epithelium. Collagen proliferation is triggered, promoting tissue contracture and scar formation. Platelets contain a rich cytoplasm including actin, myosin, glycogen, lysosomes, and dense granules and a-granules. The secretion of a-granules include proteins like platelet-derived growth factor (PDGF), which stimulates wound healing and is a powerful mitogen for vascular smooth muscle. Another important protein secreted by platelets is von Willebrand factor, which regulates circulating levels of factor VIII.

Platelets respond rapidly to tissue damage and are triggered by ADP to aggregate at the site of the injury. A positive feedback mechanism occurs as platelets begin to secrete cytokines and various proteins which in return bind to the platelet promoting further aggregation and granule release. Platelet aggregation is also induced by platelet-activating factor (PAF), a cytokine released by platelets and other immunologic proteins. PAF stimulates the secondary messenger, G-protein, which increases the production of arachidonic acid derivates, including thromboxane Az (TXA2), which serves as a weak agonist for the activation of soluble N-ethymaleimide soluble factor receptor (SNARE). The SNARE machinery is responsible for the fusion of granule to the inner leaflet of the plasma membrane, resulting in the exocytosis of essential chemokines.

The activation of platelets via agonist can promote the release of an abundant selection of chemokines including epidermal growth factor (EGF), adenosine diphosphate (ADP), fibronectin, fibrinogen, histamine, platelet-derived growth factor (PDGF), serotonin, and von Willebrand factor. The activation of platelets is preceded by granule release. The activation of platelets is most commonly done by introducing thrombin or calcium.

Recent therapeutic procedures have demonstrated benefits of harvesting platelets and, following their activation, injecting or applying them topically in the form of platelet-rich plasma at the site of an injured region of tissue. Platelet-rich plasma is a source of platelets which is derived from blood by sequestering and concentrating platelets by gradient density centrifugation. Platelet-rich plasma not only contains cytokines and growth factors, but has been demonstrated in the treated wound to induce the upregulation of vital growth factors including vascular endothelial growth factor (VEGF), fibroblast growth factor (FGF), EGF, PDGF and transforming growth factor beta (TGF-β). Growth factors are proteins that serve a mediatory role in the injury healing process, capable of stimulating cellular proliferation as well as cellular differentiation. Platelet-rich plasma has many applications such as accelerated healing of infected wounds, abscesses, sinuses and fistulae; osteogenesis; angiogenesis; alleviating chronic tendonitis, cartilage and disc regeneration; cardiac tissue repair; vascular disorders, venous stasis ulcers; necrosis, post-operative tissue damage; and acute and chronic injuries.

Undesirable conditions of the skin include wrinkles; rosacea; enlarged pores; sun damage; actinic keratoses; actinic chelitis; acne vulgaris; brown spots such as age-spots and freckles; hyper- and hypo-pigmentation; keloiding; broken blood vessels; vascular pigmented lesions, including telangiectasias (“spider veins”) and hemangiomas; scars, including hypertrophic scars, as a result of acne, trauma, burns and surgery; and unwanted hair or tattoos. As used herein, these undesirable conditions are considered injuries to the skin.

Numerous treatments have been developed to improve the appearance of the skin. While chemical peels (“chemabrasion”), dermabrasion and microdermabrasion are still popular courses of treatment in many physicians' offices, more doctors are utilizing other invasive treatments. Other such invasive treatments used to improve the skin in which portions of the skin are damaged or removed include intense pulsed light (“IPL”), radio frequency (“RF”), ablative lasers, and cryogenics.

These treatments produce some discomfort and considerable downtime for recovery. Certain types of conditions are difficult to correct without further destruction of the surrounding skin structures. Furthermore, while the end results can be beautiful, each treatment causes, to a greater or lesser degree, damage that causes the patient's skin to be unsightly for a period of time until it heals. Patients express concerns with pain during and post-recovery, as well as the length of the recovery periods. Less invasive approaches often require repeat treatments over a period of five or more months. Even these less aggressive treatments may cause the skin to become slightly pink or puffy for a day or so. The more aggressive treatments, such as with the ablative lasers, cause severe swelling, redness, bumps and blisters on or around the treated area, and sometimes crusting or scabbing. Extended healing periods are almost always involved, causing interruption in normal activities and, many times, loss of work. Patients benefit when less pain is experienced, reducing medication levels as well as minimizing post-procedure bruising while increasing tissue perfusion. It would be desirable to reduce the amount of pain and healing time. Similarly, soft tissue and musculoskeletal injuries below the skin have historically been treated with treatments that require extended healing periods, particularly in patients with diabetes, malnutrition, chronic infections and post-irradiation injury.

Light therapy using visible wavelengths has been shown to induce physiological responses in cells via the activation of specific enzymes, becoming an important instrument to treat a wide array of disorders externally. Laser therapy has been shown to suppress apoptosis; promote the proliferation of healthy viable cells; preserve membrane and genetic material of cells that are nutritionally starved; revitalize erythrocytes enhancing their oxyphoric function; enhance fertilization potential of spermatozoa; stimulate the differentiation of satellite stem cells and reduce the extent of myocardial infarctions and ischemic strokes. Laser therapy has also been shown to promote the upregulation of growth factors such as VEGF, basic fibroblast growth factor (bFGF), and numerous others; moreover, laser therapy has been shown to suppress the activation of nuclear factor Kβ and cyclooxygenase-2 thus serving as a potent anti-inflammatory.

Low-level laser therapy (LLLT) itself causes no immediate detectable temperature rise of the treated tissue, no sensation to the patient, and no macroscopically visible changes in tissue structure. Consequently, the treated and surrounding tissue is not heated and is not damaged. LLLT improves injury healing, reduces edema, and relieves pain of various etiologies, including successful application post-operatively to liposuction to reduce inflammation and pain. LLLT is also used during liposuction procedures to facilitate removal of fat by causing intracellular fat to be released into the interstice. It is also used in the treatment and repair of injured muscles and tendons. The combination of laser therapy and PRP could serve as a highly efficacious means to treat a wide-assortment of medical conditions.

It would be desirable to enhance tissue repair by treating a patient with platelet-rich plasma and low-level laser energy. The combination of the two therapies would enable the patient to heal faster and experience less pain. This can be extremely useful for procedures that require multiple treatments in order to get the desired results such as for wound healing and skin repair. For example, if the patient has less pain, then the practitioner can be more aggressive with subsequent treatments and, with faster healing, the interval between treatments will be shorter. The patient will thus achieve the desired results faster.

Therefore an object of this invention is to provide a method of treating injured tissue using PRP and low-level laser therapy. Another object of this invention is to provide a method of tissue repair using PRP and low-level laser therapy. Another object of this invention is to provide a method of treating injured tissue by activating PRP using low-level laser therapy.

### SUMMARY OF THE INVENTION

This invention is a method of treating a patient's injured tissue by applying platelet-rich plasma and laser energy to the injured tissue. The applied energy is low-level, causing no sensation to the patient. The laser energy can be applied before, after, or during the platelet-rich plasma application, or any combination thereof. Additional laser therapy may be applied over the entire extremity containing the injury, any non-injured adjacent tissue, as well as to the patient's entire body for stimulation of other body systems. Additionally, laser energy can be applied to the platelet-rich plasma before the platelet-rich plasma is applied to the patient.

### BRIEF DESCRIPTION OF THE DRAWINGS

FIG. 1 illustrates laser energy being applied to platelet-rich plasma.

FIGS. 2 A and B illustrate the method applied to a wound on a hand.

FIGS. 3 A and B illustrate the method applied to a knee injury.

### DETAILED DESCRIPTION OF THE INVENTION A patient's injured tissue is treated using platelet-rich plasma (“PRP”) in combination with low-level laser therapy. As used herein, injured tissue includes tissue that has been harmed as well as tissue that has an undesirable characteristic but is not necessarily considered harmed, such as tissue covered with freckles.

PRP has a concentration of platelets greater than the peripheral blood concentration suspended in a solution of plasma, with typical platelet counts ranging from 500,000 to 1,200,000 per cubic millimeter, or even more. PRP is formed from the concentration of platelets from whole blood, and may be obtained using autologous, allogenic, or pooled sources of platelets or plasma. PRP may be formed from a variety of animal sources, including human sources. Preferably PRP used to treat a patient is autologous.

PRP can be isolated by a variety of methods. For example, approximately 40-120 milliliters of venous blood is collected from the cubital vein. The whole blood is collected into a into a sterile container, such as a Vacuette®, with citrate and centrifuged for approximately 8 minutes at 3,000 rpm using a standard centrifuge. The centrifugation process results in two fractions: a red, opaque lower fraction consisting of red and white blood cells and platelets, called the blood cell component, and a second upper straw-yellow turbid fraction with plasma and platelets, called the serum component. The top yellow portion of the serum component contains autologous fibrinogen and is very low concentration of platelets (platelet-poor plasma). The portion around the transition between the serum component and blood cell component has the highest concentration of platelets. Using a standard pipette, the platelet-rich region is removed and placed into a sterile container without citrate. This pipetted material is centrifuged again for approximately 6 minutes at 1,000 rpm using a standard centrifuge.

After the second centrifugation, two fractions will again be obtained. The top fraction is yellow serum with fibrinogen and has a very low concentration of platelets. The remaining substance is the available platelet concentrate, rich in platelets with autologous fibrinogen. This region is removed via standard pipetting technique, and placed into sterile container. Other methods, many known in the art, can be used to isolate PRP.

40 ml of blood is capable of producing about 4 ml of platelet-enriched gel, thus 1 ml of platelets can be extracted per 10 ml of blood. Therefore, if more than 4 ml of platelet-enriched gel is required, more than 40 ml of whole venous blood must be extracted.

Once the PRP has been isolated, it can be mixed into various forms including injectable and topical solutions. For an injectable solution, the PRP can be mixed with normal saline or left in its isolated concentrate form. The PRP can be combined with other injectable components including hyaluronic acid, anti-oxidants, steroids and other growth factors, and other components used for therapeutic purposes. For a topical solution, the PRP can be applied as the isolate, as a gel, mixed with saline or combined with other components including hyaluronic acid, anti-oxidants, steroids and other growth factors, and other components used for therapeutic purposes. The pH for the injectable or topical solution can be modified by adding sodium bicarbonate.

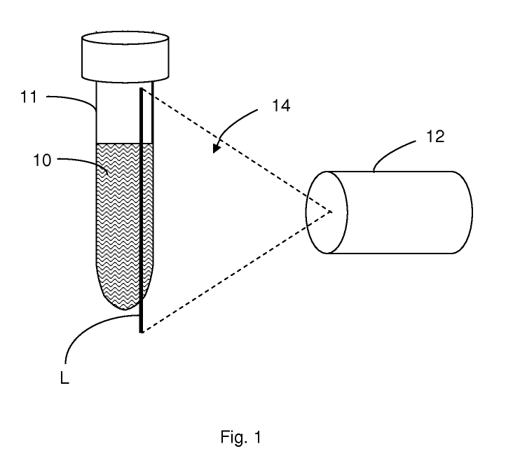

Optionally, once the PRP has been isolated it can be treated with low-level laser energy to prior to its application to the patient to stimulate the PRP and to activate growth factors and other beneficial components. Devices for applying laser energy are known in the art, and are further described below. The PRP can be laser treated as the isolate, or in its injectable or topical form. For example, laser energy 14 can be applied to the PRP isolate 10 in the sterile container 11 from a laser energy source 12. See FIG. 1. The laser energy applied has a wavelength between about 400 nm to 1500 nm, in a constant wave, pulsed, or a combination of both. Pulse frequencies from 0 to 100,000 Hz may be employed. The output power of the laser used to apply the laser energy is between about 1 mW to 500 mW. The laser energy is applied long enough to the PRP is to stimulate the platelets, which usually takes between about 1 second to about 27.8 hours. Once laser stimulation is complete, the PRP can be either topically applied or injected. Further, this activated PRP solution can be used without subsequent low-level laser therapy for wound repair, skin rejuvenation, wrinkle reduction, and treatment of numerous dermatological disorders.

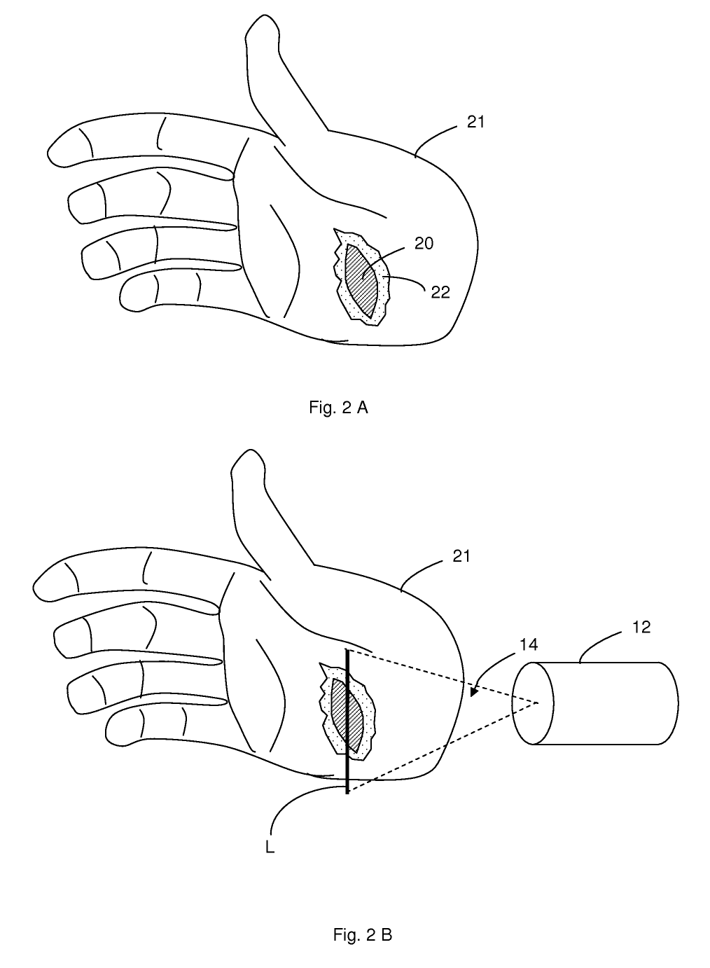

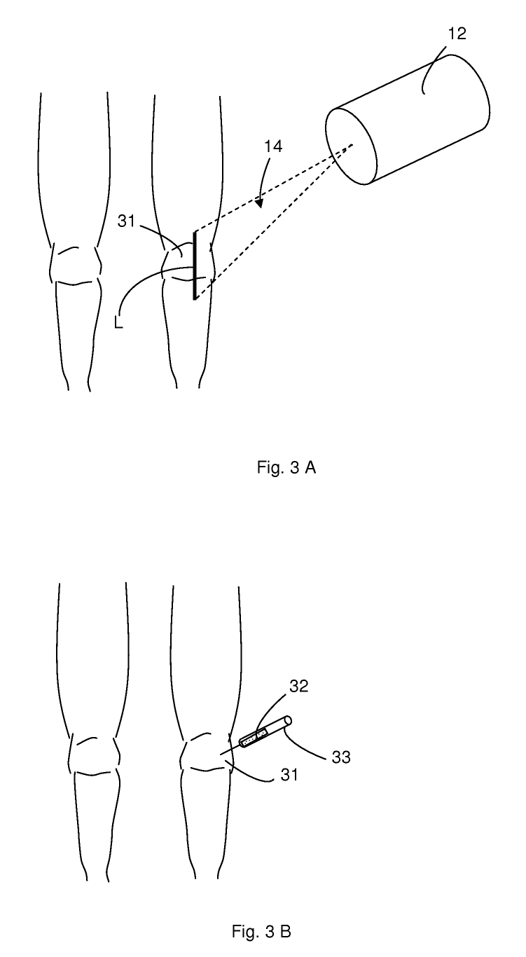

PRP, whether or not treated with laser energy, is applied to the injury. Typically the PRP is applied directly to the injury, either topically or by injection into the injured site. For example, FIG. 2A illustrates PRP gel 22 applied topically to a wound 20 on the palm of a hand 21. The PRP can be injected into deep tissue structures to treat orthopedic conditions, repair ligaments and tendons, promote bone growth, or repair vascular issues and non-healing medical disorders. For example, FIG. 3B illustrates PRP 32 applied by injection to an injured ligament (not shown) within a knee 31. Further, the injection can be superficial for the treatment of non-healing wounds, dermatologic skin disorders, skin rejuvenation, wrinkle reduction, and for anti-aging purposes.

Laser energy can be applied to the patient before, after, or during the platelet-rich plasma application, or any combination thereof. The timing of the application of laser energy relative to the application of platelet-rich plasma will depend on a number of factors, including the type of injury, the location of the injury, and pragmatic considerations such as the number of practitioners present or the amount of operating space. That is, laser energy can be applied before, after or during the application of platelet-rich plasma, or any combination of timing. In the preferred method, the laser energy is applied at least after the PRP is applied, and more preferably promptly after the PRP is applied. Additional laser energy may be applied over the entire extremity containing the injury, any non-injured adjacent tissue, as well as entire body application for stimulation of other body systems such as the lymphatic, circulatory, and nervous systems.

Low-level laser energy serves as a potent agonist to activate the platelets, thereby enhancing the efficacy of PRP and eliminating unwanted effects of chemical activators such as thrombin or calcium.

There are a number of variables in determining sufficient and appropriate laser therapy including the wavelength of the laser beam, the area impinged by the laser beam, laser energy, pulse frequency, treatment duration, depth and type of the injury, and tissue characteristics. The wavelength of the applied laser energy depends on the nature of the injury, among other factors, and ranges from ultraviolet to infrared. Preferably, however, the applied laser energy is in the visible spectrum, from about 396 nm to about 800 nm. Pulse frequencies from 0 to 100,000 Hz may be employed to achieve the desired effect on the patient's tissue. When there are no pulses, a continuous beam of laser light is generated. The patient feels no sensation of the low-level laser energy being applied.

Low-level lasers, such as those described in U.S. Pat. Nos. 6,013,096, 6,746,473, and 7,118,588 which are incorporated herein by reference, can be used for treating injuries with the present method. Hand-held lasers are particularly convenient for treating areas at or near the injury on a patient's body, while stand-alone lasers are convenient for applying laser energy to PRP storage containers.

In the preferred embodiment, a hand-held laser device is used to apply laser energy to the patient. The laser device has at least one energy source, preferably a semiconductor laser diode that produce light in the red range of the visible spectrum, having a wavelength of about 635 nm. The laser device includes a rod lens through which the laser light is emitted, creating a line of light L. See FIGS. 2B and 3A. The line of light L is scanned across the area of injury. Alternatively, as explained in more detail in U.S. Pat. No. 7,118,588, the laser device includes a carriage that rotates about an axis that is substantially co-axial to the incident laser beam, thereby causing the laser energy passing through the optical element to sweep through a 360° circle, resulting in a large circular beam spot. The carriage is rotated with a drive assembly. The drive assembly is preferably a main drive gear which is mated with a minor drive gear. The minor drive gear is driven by a main drive motor. The carriage rotates around the axis as the main drive gear is turned. Thus, the laser beam from laser energy source passes through a hollow spindle and strikes an optical element which deflects the laser beam into a linear beam spot L that, in combination with the rotation, appears as a circular beam spot. Preferably, the laser beam remains coaxial with the hollow spindle through the optical element, so that the center of the beam spot created by the optical element is on the axis of the hollow spindle.

### Example 1

A traumatic hand wound 20 is treated with a combination of PRP and laser energy. Following standard debridement and wound toilet, a volume of PRP gel 22 sufficient to fill the wound cavity is applied topically to the wound 20. See FIG. 2A. The wound 20 is then treated with low-level laser energy 14. See FIG. 2B. Between 0.2 and 5 joules of laser energy is provided by the laser energy source. In this example, the laser energy 14 is provided by a laser source 12 that emits a line of laser light L that is passed repeatedly across the wound 20. Application of laser energy beyond the periphery of the wound is acceptable, and improves healing. A laser device is used that emits under 1 watt having a wavelength of 635 nm. Laser energy treatment is repeated daily to the PRP-filled wound for 5 days, following which, a repeat application of PRP is applied and the cycle repeated. At each subsequent PRP application, the volume required steadily decreases as the wound contracts and heals.

### Example 2

A combination of low level laser therapy and PRP is used to treat a patient's strained knee ligament. The entire knee 31 is treated externally with laser energy 14 provided by a laser source 12. See FIG. 3A. Between 0.2 and 5 joules of laser energy is provided by the laser energy source that emits a line of laser light L. A laser device is used that emits under 1 watt having a wavelength of 635 nm. Subsequently, PRP 32 is injected into the knee using a syringe 33, as deep as necessary to be near the injury to the ligament. See FIG. 3B.

### Example 3

Diabetic wounds, surgically induced, traumatic or infective, are treated with a combination of laser energy and PRP. Following standard debridement and wound toilet, the wound is treated with low-level laser energy. Between 0.2 and 5 joules of laser energy is provided by a laser device of under 1 watt having a wavelength of 635 nm. Then, a volume of PRP sufficient to fill the wound cavity is applied topically to the wound. Subsequently, the laser energy treatment is repeated daily to the PRP-filled wound for 5-7 days, following which, a repeat application of PRP is applied and the cycle repeated. At each subsequent PRP application, it will be noted that the volume required steadily decreases as the wound contracts and heals.

The observable biological effect following low-level laser therapy transpires in the presence of a photoacceptor molecule, a molecule able of absorbing the photonic energy emitted. A molecule capable of such absorption generally contains a transition metal. Transition metals are characterized by their incomplete d orbitals. The unique ability to absorb light radiation is directly attributed to the distinctive electron configuration of transition metals.

Transition metals are also found in cytochrome c oxidase. Cytochrome c oxidase is a key enzyme in cellular respiration, the electron transfer chain that results in the production of adenosine triphosphate (“ATP”). ATP provides the energy used to promote cell growth. Thus, the likely mechanism for the enhanced healing using the combination of platelet-rich plasma and low-level laser energy is the excitation of the transition metals in cytochrome c oxidase, which stimulates the platelets to generate more growth factors such as platelet-derived growth factor (PDGF) and transforming growth factor beta (TGF-β), thus speeding wound healing.

An additional mechanism for the enhanced healing using the combination of platelet-rich plasma and low-level laser energy is the effect laser radiation has on the intracellular redox potential. Many cellular signaling pathways are regulated by the intracellular redox state, including those signaling pathways that control gene expression. A shift towards a more oxidative state stimulates various cellular signaling systems and low-level laser therapy has been shown to promote a transient shift in the intracellular redox potential in the way of greater oxidation.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. A method of treating a patient's injured tissue comprising:
a) applying in combination platelet-rich plasma to the injured tissue and laser energy at or near the injured tissue.

2. The method of claim 1 wherein the laser energy is applied to the patient after the platelet-rich plasma is applied.

3. The method of claim 2 wherein the laser energy applied to the patient is equal to or less than about 5 joules.

4. The method of claim 1 wherein the laser energy has a wavelength in the visible spectrum.

5. The method of claim 1 wherein the injured tissue is dermal.

6. The method of claim 1 wherein the injured tissue is musculoskeletal.

7. The method of claim 1 wherein the platelet-rich plasma comprises platelets obtained from the patient.

8. The method of claim 1 wherein the platelet-rich plasma is applied topically to the patient.

9. The method of claim 1 wherein the platelet-rich plasma is injected into the patient.

10. The method of claim 1 further comprising identifying a patient with an undesirable skin condition wherein the injured tissue has been injured as a result of the patient having the undesirable skin condition.

11. The method of claim 1 further comprising identifying a patient with diabetes wherein the injured tissue has been injured as a result of the patient having diabetes.

12. The method of claim 1 further comprising:
a) applying additional laser energy to platelet-rich plasma prior to applying the platelet-rich plasma at or near the injured tissue.

13. The method of claim 1 further comprising:
a) applying additional laser energy to the patient not at or near the injured tissue.

14. The method of claim 1 wherein the laser energy is applied at or near the injured tissue prior to applying the platelet-rich plasma at or near the injured tissue.

15. The method of claim 1 wherein the laser energy is applied at or near the injured tissue after to applying the platelet-rich plasma at or near the injured tissue.

16. The method of claim 1 wherein the laser energy is applied at or near the injured tissue simultaneously with applying the platelet-rich plasma at or near the injured tissue.

17. A method of treating a patient's injured tissue comprising:
a) applying platelet-rich plasma at or near the injured tissue; and
b) applying therapeutic laser energy to the patient.

18. A method of treating a patient's injured tissue comprising:
a) isolating platelet-rich plasma;
b) applying laser energy to platelet-rich plasma; and
c) applying laser-treated platelet-rich plasma at or near the injured tissue.

19. The method of claim 18 further comprising applying laser energy to the patient.

20. The method of claim 18 wherein the platelet-rich plasma is isolated from the patient's blood.
